(19) United States
(12) Patent Application Publication
(10) Pub. No.: US 2020/0275872 A1
(43) Pub. Date: Sep. 3, 2020

(54) BRAIN FUNCTION MEASUREMENT APPARATUS

(71) Applicant: SHIMADZU CORPORATION, Kyoto-shi (JP)

(72) Inventors: Ryu Konoshita, Kyoto-shi (JP); Rintaro Yamamoto, Kyoto-shi (JP)

(73) Assignee: SHIMADZU CORPORATION, Kyoto-shi (JP)

(21) Appl. No.: 16/288,306

(22) Filed: Feb. 28, 2019

(51) Int. Cl. A61B 5/1455; A61B 5/00

(52) U.S. Cl. CPC A61B 5/14553; A61B 5/6814; A61B 2562/04; A61B 2562/185; A61B 2562/0238

(57) ABSTRACT

The brain function measurement apparatus comprises a plurality of measurement probes; a holder which is fitted to the head of a subject and comprises a plurality of probe attachment parts; a light blocking member which is arranged so as to cover the holder and blocks light from entering the plurality of probes; and a contact prevention member which prevents the light blocking member from contacting the measurement probes.

### CROSS-REFERENCE TO RELATED APPLICATIONS

The related application number JP2016-164089, Brain function measurement apparatus, Aug. 24, 2016, Ryu Konoshita and Rintaro Yamamoto, upon which this patent application is based, is hereby incorporated by reference.

### BACKGROUND OF THE INVENTION

### Field of the Invention

The present invention relates to a brain function measurement apparatus, particularly, a brain function measurement apparatus comprising a plurality of measurement probes arranged on the head of a subject.

### Description of the Prior Art

Brain function measurement apparatuses comprising multiple measurement probes arranged on the head of a subject are known in the prior art. Such a brain function measurement apparatus is disclosed, for example, in Japanese Unexamined Patent Application Publication 2015-33561.

The aforementioned Japanese Unexamined Patent Application Publication 2015-33561 discloses a brain function measurement apparatus comprising a plurality of measurement probes which are arranged on the head of a subject. This brain function measurement apparatus is provided with a holder which is fitted to the head of a subject and has a plurality of attachment parts. The holder is designed to have measurement probes inserted into any one of the plurality of attachment parts. The brain function measurement apparatus is furthermore designed such that measurement light is applied to the head of the subject from one of a plurality of measurement probes, and measurement light scattered or reflected from inside the brain in the subject's head is received by the other measurement probes.

Here, in a conventional brain function measurement apparatus as described in aforementioned Japanese Unexamined Patent Application Publication 2015-33561, when a measurement probe is to be arranged in a hair-covered area (a site where there is hair) on the subject's head, the head surface is exposed to the attachment part of the holder by parting the hair using a rod-shaped tool, so that the hair will not interfere with the measurement light. After measurement probes have been attached to the attachment parts, if the magnitude of the signal acquired by the brain function measurement apparatus is not within a specified range, correction of the installation state (installation angle, etc.) of the measurement probes may be performed, or an operation may be performed whereby some of the measurement probes are temporarily removed, the hair is again parted using a rod-shaped tool, and then the measurement probe is reinserted into the attachment part. The above-described operations to ensure that the magnitude of the signal acquired by the brain function measurement apparatus is within the specified range will be referred to hereinafter as “adjustment operations”.

Moreover, there are cases where the brain function measurement apparatus as described in aforementioned Japanese Unexamined Patent Application Publication 2015-33561 may be used, for example, at locations exposed to sunlight, locations exposed to illumination light, locations where light is emitted by another measurement instrument, and the like. In such cases, if light other than the measurement light enters the measurement probes, it will become a source of noise. In this connection, one may consider attaching a light blocking member to cover the measurement probes after attaching the measurement probes to the holder, so as to prevent sunlight, illumination light or other external light from entering the measurement probes.

However, when a light blocking member is attached to cover the measurement probes in a conventional brain function measurement apparatus such as that of aforementioned Japanese Unexamined Patent Application Publication 2015-33561, the light blocking member will contact the measurement probes, which may alter the installation state of the measurement probes, making it impossible to correctly measure the signal. In such cases, it becomes necessary to perform the aforementioned adjustment operations again, so the efficiency of the measurement preparation operations for the brain function measurement apparatus decreases. Therefore, a brain function measurement apparatus has been desired, which would make it possible to prevent light other than measurement light from entering the measurement probes while avoiding reduction in efficiency of measurement preparation operations.

### SUMMARY OF THE INVENTION

This invention was made to resolve the problem described above. It is an object of this invention to provide a brain function measurement apparatus capable of preventing light other than measurement light from entering the measurement probes while avoiding reduction in the efficiency of measurement preparation operations.

To achieve the aforesaid object, the brain function measurement apparatus according to one aspect of this invention comprises: a plurality of measurement probes which either apply measurement light to the head of a subject or receive measurement light from the head of the subject; a holder which is fitted to the head of the subject and comprises a plurality of probe attachment parts for attaching the plurality of measurement probes; a light blocking member which is arranged so as to cover the holder in a state where the plurality of measurement probes have been attached to the probe attachment parts, and blocks light from entering the plurality of measurement probes; and a contact prevention member which is arranged between the holder and the light blocking member and prevents the light blocking member from contacting the measurement probes.

In the brain function measurement apparatus according to this aspect of the invention, as described above, the brain function measurement apparatus is provided with a light blocking member which is arranged so as to cover the holder in a state where a plurality of measurement probes have been attached to the probe attachment parts, and blocks light from entering the plurality of measurement probes; and a contact prevention member which is arranged between the holder and the light blocking member and prevents the light blocking member from contacting the measurement probes. This makes it possible to prevent light other than measurement light from entering the measurement probes by means of the light blocking member. Furthermore, the contact prevention member prevents the light blocking member from contacting measurement probes on which adjustment operations have been performed, so even if a light blocking member is attached, the installation state of the measurement probes will not change, so repeated measurement probe adjustment operations can be avoided. Consequently, due to the fact that the installation state of the measurement probes does not change, measurement light from the measurement probes can be accurately applied to the head of the subject, and light other than measurement light can be prevented from entering the measurement probes while avoiding reduction in efficiency of measurement preparation operations.

The brain function measurement apparatus according to the above aspect is preferably configured such that the height of protrusion of the contact prevention member from the holder in the state where the contact prevention member has been arranged on the holder is greater than the height of protrusion of the measurement probes from the holder. Configuring the apparatus in this manner makes it possible to increase the distance between the light blocking member and the measurement probes by means of the contact prevention member, whereof the height of protrusion from the holder is relatively large, thus making it possible to more reliably prevent the light blocking member from contacting the measurement probes.

In the brain function measurement apparatus according to the above aspect, the contact prevention member preferably comprises dummy measurement probes which are removably attached to the probe attachment parts. If the apparatus is configured in this manner, there is no need to separately provide attachment parts for attaching the contact prevention member to the holder. Furthermore, configuring the dummy measurement probes to be removably installable in the probe attachment parts makes it possible to attach the dummy measurement probes to probe attachment parts corresponding to the optimal locations for attaching dummy measurement probes in accordance with the state of arrangement of measurement probes.

In this case, the total length of the dummy measurement probes is preferably made adjustable. Such a configuration allows the total length to be appropriately adjusted according to the location of arrangement of the dummy measurement probe such that the light blocking member will not contact the measurement probes, thus making it possible to more effectively prevent the light blocking member from contacting the measurement probes.

In the brain function measurement apparatus according to the above aspect, preferably, the light blocking member is formed as a flexible light blocking cloth, the light blocking cloth is arranged such that the edge of the light blocking cloth will fit tightly against the forehead of the subject upon tying a pair of string members connected to two ends of the light blocking cloth; and the contact prevention member is either arranged on the holder between the edge of the light blocking cloth and the measurement probes, or is arranged on the holder at a position adjacent to those measurement probes that are arranged toward the edge of the light blocking cloth. Here, when the light blocking member is formed as a light blocking cloth, it becomes possible to bring the edge of the light blocking cloth into tight contact with the forehead by tying two ends of the light blocking cloth with string members, in order to block light other than measurement light from entering the measurement probes from the direction of the subject's forehead. In this case, the closer to the edge of the light blocking cloth, the closer the light blocking cloth is arranged to the surface of the subject's forehead, so the light blocking cloth will more readily contact those measurement probes which are closer to the edge of the light blocking cloth. Noting this point, in the present invention, the contact prevention member is either arranged on the holder between the edge of the light blocking cloth and the measurement probes, or is arranged on the holder at a position adjacent to those of the plurality of measurement probes which are arranged toward the edge of the light blocking cloth. A contact prevention member arranged at a position as described above thus makes it possible to more effectively prevent the measurement probes which are relatively more prone to contacting the light blocking cloth from contacting the light blocking cloth.

In the brain function measurement apparatus according to the above aspect, preferably, the contact prevention member comprises a hair parting part for parting hair on the head of the subject. Designing the apparatus in this manner makes it possible to use the contact prevention member as a hair parting member, thereby eliminating the need to provide a member for hair parting separately from the contact prevention member. As a result, an increase in the number of parts of the brain function measurement apparatus can be avoided even though a contact prevention member is provided.

In this case, preferably, the contact prevention member comprises a light source part which emits light; and the hair parting part is designed to guide light from the light source part in the state where the contact prevention member is detached from the holder, thereby allowing the light from the light source part to be applied to the head of the subject. When hair on the subject's head is to be parted, designing the apparatus in this manner makes it possible to apply light from the light source to the area where hair is being parted. As a result, the efficiency of the operation of parting hair can be increased.

### BRIEF DESCRIPTION OF THE DRAWINGS

FIG. 1 is a drawing schematically illustrating the overall configuration of a brain function measurement apparatus according to a first embodiment.

FIG. 2 is a block diagram illustrating the configuration of the brain function measurement apparatus according to a first embodiment.

FIG. 3 is an overall perspective view illustrating the state where measurement probes and dummy probes of the brain function measurement apparatus according to a first embodiment have been arranged in the holder.

FIG. 4 is a perspective view illustrating the configuration of the measurement probes and dummy probes of the brain function measurement apparatus according to a first embodiment.

FIG. 5 is a partial perspective view illustrating the state where the measurement probes and dummy probes of the brain function measurement apparatus according to a first embodiment have been arranged in the holder.

FIG. 6 is a drawing illustrating the light blocking cloth of the brain function measurement apparatus according to a first embodiment.

FIG. 7 is a drawing intended to explain the arrangement of measurement probes and dummy probes in the holder of the brain function measurement apparatus according to a first embodiment.

FIG. 8 is a partial perspective view illustrating the state where measurement probes and dummy probes of the brain function measurement apparatus according to a second embodiment have been arranged in the holder.

FIG. 9 is a drawing intended to explain the configuration of the hair parting part of a dummy probe of the brain function measurement apparatus according to a second embodiment.

FIG. 10 is a drawing intended to explain the arrangement of measurement probes and dummy probes in the holder of the brain function measurement apparatus according to a first modified example of the first embodiment.

FIG. 11 is a perspective view illustrating the configuration of a dummy probe according to a second modified example of the second embodiment.

FIG. 12 is a perspective view illustrating the configuration of a dummy probe of a brain function measurement apparatus according to a third modified example of the second embodiment.

### DESCRIPTION OF PREFERRED EMBODIMENTS

Specific embodiments of the present invention will be described below based on the drawings.

### First Embodiment

### Overall Configuration of Brain Function Measurement Apparatus

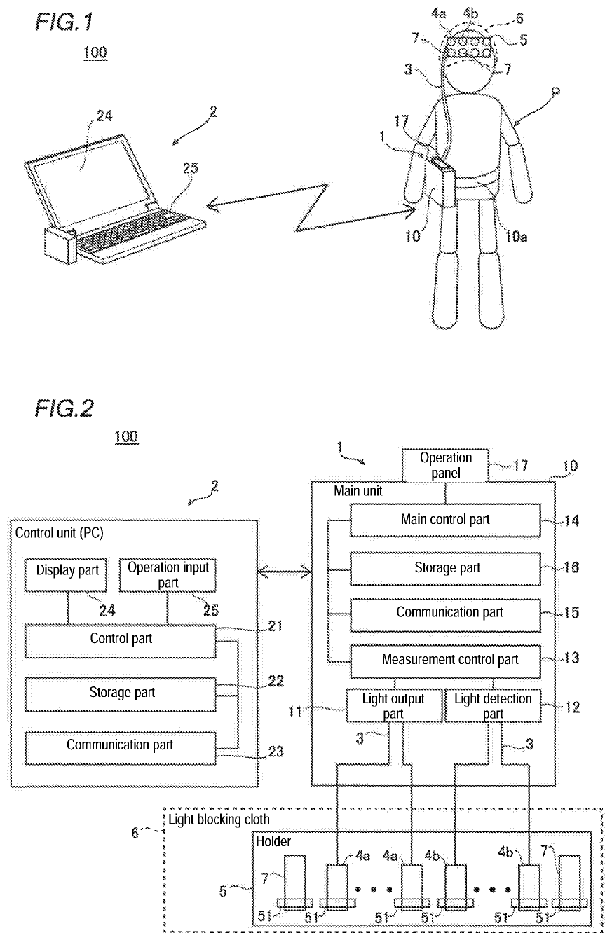

First, the overall configuration of a brain function measurement apparatus 100 according to a first embodiment of the present invention will be described with reference to FIGS. 1 through 7.

As shown in FIG. 1, the brain function measurement apparatus 100 according to the first embodiment is an optical measurement device which non-invasively optically measures the brain activity of the subject P, and is configured as a brain function imaging device which visualizes the state of activity near the brain surface in real time based on functional near-infrared spectroscopy (fNIRS). Furthermore, the brain function measurement apparatus 100 comprises, for example, a portable main unit 1 which performs brain function measurement while being carried by the subject P, and a control unit 2 which receives the brain function measurement data generated by the main unit 1 via wireless communication and records this data.

Thus, with the brain function measurement apparatus 100 according to the first embodiment, the subject P is not constrained to being in the vicinity of the measurement apparatus (control unit 2) during brain function measurement and can move freely while carrying the main unit 1, making it possible to perform brain function measurement in an environment closer to daily life. For example, with the brain function measurement apparatus 100 according to the first embodiment, it is possible to perform brain function measurement on a subject P whole is active outdoors and exposed to the sun, or to perform brain function measurement on a subject P who is moving indoors through an area where other measurement devices which emit light are installed.

As shown in FIG. 1 and FIG. 2, the main unit 1 comprises a light output part 11, light detection part 12, a measurement control part 13, a main control part 14, a communication part 15 and a storage part 16 inside a case 10 of a portable size. Furthermore, the main unit 1 is provided with an operation panel 17 (see FIG. 1) in a portion of the surface of the case 10.

Furthermore, the main unit 1 comprises light sending probes 4a and light receiving probes 4b, connected via optical fibers 3. Furthermore, the main unit 1 is designed to allow connection of multiple (for example, four) light sending probes 4a and multiple (for example, four) light receiving probes 4b. Furthermore, the main unit 1 can be carried by the subject P by securing to the waist or the like using a harness 10a. It will be noted that the light sending probes 4a and light receiving probes 4b are examples of “measurement probes” of the present invention. The light sending probes 4a and light receiving probes 4b will be referred to hereinafter as measurement probes 4 when their configurations are not being specially distinguished.

Furthermore, as shown in FIG. 2, the brain function measurement apparatus 100 comprises a holder 5, which is fitted to the head of a subject P and comprises a plurality (for example, a number greater than the number of measurement probes 4) of attachment parts 51 for attaching light sending probes 4a and light receiving probes 4b. The light sending probes 4a and light receiving probes 4b are arranged on the surface of the head of the subject P by attaching to the holder 5 which has been fitted to the head of the subject P. It will be noted that attachment units 51 are an example of the “probe attachment units” referred to in the patent claims.

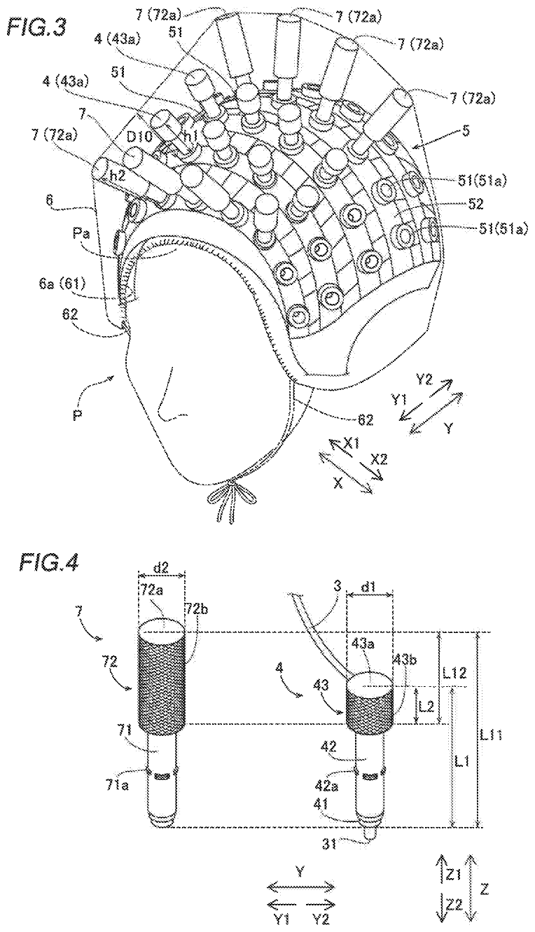

Furthermore, as shown in FIG. 3, in the first embodiment, the brain function measurement apparatus 100 comprises a light blocking cloth 6. The light blocking cloth 6 is arranged so as to cover the holder 5 in a state where measurement probes 4 have been attached to attachment parts 51, and is designed to block light from entering the measurement probes 4. Namely, the light blocking cloth 6 makes it possible to prevent sunlight from entering the measurement probes 4 even when the brain function measurement apparatus 100 is used outdoors. It will be noted that the light blocking cloth 6 is an example of the “light blocking member” referred to in the patent claims.

Furthermore, in the first embodiment, the brain function measurement apparatus 100 comprises dummy probes 7. The dummy probes 7 are arranged between the holder 5 and the light blocking cloth 6 and are designed to prevent the light blocking cloth 6 from contacting the measurement probes 4. It will be noted that the dummy probes 7 are an example of the “contact prevention member” and “dummy measurement probes” referred to in the patent claims.

Furthermore, the light sending probes 4a are designed to apply measurement light to the head of the subject P when arranged in the holder 5. Moreover, the light receiving probes 4b are designed to receive measurement light from the head of the subject P when arranged in the holder 5. Namely, the main unit 1 is designed to emit measurement light in the near-infrared wavelength range through the light sending probes 4a and detect measurement light which has been reflected or scattered from inside the head of the subject P by allowing it to enter into the light receiving probes 4b, thereby acquiring the intensity (received amount) of measurement light.

In particular, as shown in FIG. 2, the light output part 11 is designed to output measurement light to the light sending probes 4a via optical fibers 3. The light output part 11 comprises, for example, a semiconductor laser or light emitting diode, and is designed to allow output of measurement light of multiple wavelengths (for example, measurement light of three wavelengths—780 nm, 805 nm and 830 nm) in the high bio-penetration near-infrared wavelength region. The light detection part 12 comprises, for example, a photomultiplier tube or photodiode, and is designed to acquire and detect, through optical fiber 3, measurement light which has entered a light receiving probe 4b. The light detection part 12 outputs an electrical signal corresponding to the detected measurement light.

The main unit 1 is furthermore designed to acquire changes in hemoglobin levels (oxygenated hemoglobin, deoxygenated hemoglobin and total hemoglobin) during brain activity based on the intensity of the acquired measurement light. The brain function measurement apparatus 100 can thereby non-invasively acquire changes in hemoglobin levels during brain activity, namely, changes in blood flow rate and the state of activation of oxygen metabolism. Furthermore, the apparatus is designed to acquire two-dimensional distributions, with optical measurement being performed to measure brain activity at each measurement point (measurement channel), consisting of a pair of light sending probe 4a and light receiving probe 4b.

Moreover, the measurement control part 13 controls the operation of light output part 11 and light detection part 12 in accordance with measurement conditions set by main control part 14, and measurement parameters relating to output intensity of measurement light, detection sensitivity of light detection part 12, etc. The main control part 14 is a computer comprising a CPU, memory, etc., which controls the measurement control part 13, communication part 15 and storage part 16 by executing a measurement program stored in storage part 16, to perform measurement operations, computation of measurement data based on obtained received light amount signals, and transmission of measurement data to the control unit 2. Furthermore, the main control part 14 is designed to receive input operations from the subject P via the operation panel 17.

The communication part 15 comprises a wireless communication module and enables bidirectional wireless communication with communication part 23 of control unit 2, described below. The storage part 16 comprises, for example, nonvolatile memory, and stores measurement conditions and programs to be executed by main control part 14.

Next, as shown in FIG. 2, the control unit 2 is a computer (PC) comprising a control part 21 consisting of a CPU, memory, etc., a storage part 22 consisting of an HDD, etc., and a communication part 23 consisting of a wireless communication module (or externally connected wireless communication unit), which computer functions as the control unit 2 of the brain function measurement apparatus 100 through execution by the control part 21 of a measurement program stored in the storage part 22. Furthermore, the control unit 2 comprises a display part 24 consisting of a liquid crystal display, etc., and an operation input part 25 consisting of a keyboard and mouse, etc.

### Configuration of Measurement Probes

As shown in FIG. 4, the measurement probe 4 is formed, for example, so as to have a cylindrical shape. The measurement probe 4 comprises a fiber head part 41, probe main body part 42 and grip part 43. Furthermore, the measurement probe 4 has a total length L1. The total length L1 is assumed to signify the distance from the lower end face of the fiber head part 41 (the end face in the direction of arrow Z2) to the upper end part 43a of the grip part 43.

Furthermore, as shown in FIG. 5, the measurement probe 4 is designed to extend in a direction (arrow Z1 direction) substantially perpendicular to the holder 5 (head) when arranged in a mounting part 51 of the holder 5. The measurement probe 4 has a protrusion height h1 from the holder 5. The protrusion height h1 is assumed to signify the distance from the upper end face of the attachment part 51 (the end face in the arrow Z1 direction) to the upper end part 43a (the end face in the arrow Z1 direction) of the grip part 43 of the measurement probe 4.

The fiber head part 41 is arranged more towards the subject's head (in the arrow Z2 direction) than the attachment part 51 in the state where the measurement probe 4 has been arranged in an attachment part 51 of the holder 5. Furthermore, the fiber head part 41 is designed to retain an optical fiber 3 in a state with the end part 31 of the optical fiber 3 exposed. The end part 31 is designed to allow the output or input of measurement light.

Optical fiber 3 is furthermore inserted inside the probe main body part 42. Furthermore, as shown in FIG. 4, the probe main body part 42 is provided with an engagement part 42a capable of engaging with an attachment part 51 of the holder 5. Specifically, the engagement part 42a is formed as a convex part capable of fitting into the concave part of engagement part 51a of attachment part 51.

Grip part 43 is provided on the side of the measurement probe 4 in the arrow Z1 direction. The surface of the side face 43b of the grip part 43 is, for example, knurled. Furthermore, the optical fiber 3 is arranged inside the measurement probe 4 via the side face 43b of the grip part 43. Namely, the measurement probe 4 is configured as an L-shaped attachment member (attachment). Furthermore, the grip part 43 has a length L2 in the Z axis direction and has a diameter d1 when viewed from the direction of arrow Z1.

### Configuration of Dummy Probe

As shown in FIG. 4, the dummy probe 7 has an outer shape similar to measurement probe 4. Specifically, the dummy probe 7 has a cylindrical shape and comprises a probe main body part 71 and grip part 72. For example, the total length L11 of the dummy probe 7 is greater than the total length L1 of the measurement probe 4 in the direction (Z axis direction) perpendicular to the plane (head surface) in which the holder 5 is arranged. It will be noted that total length L11 is assumed to signify the distance from the lower end (the end in the arrow Z2 direction) of the probe main body part 71 to the upper end 72a (the end in the arrow Z1 direction) of the grip part 72.

The probe main body part 71 has the same shape as the probe main body part 42 of a measurement probe 4. However, unlike the probe main body part 42 of a measurement probe 4, the probe main body part 71 does not have an optical fiber 3 inserted into it. Furthermore, the grip part 72 is knurled similarly to the grip part 43 of a measurement probe 4. Moreover, the grip part 72 has a length L12 in the Z axis direction. For example, the length L12 of the grip part 72 is made equal to or greater than the length L2 of the grip part 43 of a measurement probe 4. It will be noted that in FIG. 4, length L12 is illustrated as being greater than length L2.

Here, in the first embodiment, the total length L11 of the dummy probe 7 is made adjustable. Namely, the total length L11 of the dummy probe 7 is made adjustable by changing the magnitude of the aforementioned length L12 of the grip part 72 (for example, by replacing with a grip part 72 of a different length). Therefore, the relative size relationship between the total length L11 of the dummy probe 7 and the length L12 of the grip part 72 on the one hand and the total length L1 of the measurement probe 4 and the length L2 of the grip part 43 on the other, is not limited to that illustrated in FIG. 4. For example, depending on the position of arrangement of the dummy probe 7, total length L11 may be smaller than total length L1.

Furthermore, the diameter d2 of the dummy probe 7 (grip part 72) when viewed from the arrow Z1 direction is equal to or greater than the diameter d1 of measurement probe 4 (grip part 43). It will be noted that in the example of FIG. 4, the diameter d2 of the grip part 72 is shown as having approximately the same size as diameter d1 of grip part 43.

Furthermore, in the first embodiment, the dummy probe 7 is designed to be installable in and removable from mounting part 51. Specifically, the probe main body part 71 of dummy probe 7 is provided with an engagement part 71a. The engagement part 71a is designed to have the same structure as engagement part 42a of measurement probe 4.

Furthermore, as shown in FIG. 5, the dummy probe 7 is designed to extend in a direction (arrow Z1 direction) substantially perpendicular to the holder 5 (head) when arranged in the holder 5. Furthermore, the dummy probe 7 has a protrusion height h2 from the holder 5. Here, in the first embodiment, the protrusion height h2 of the dummy probe 7 is greater than the protrusion height hl of measurement probe 4. It will be noted that protrusion height h2 is used to signify the distance from the upper end face of attachment part 51 to the upper end 72a of grip part 72.

As a result, when the light blocking cloth 6 is arranged in a state where measurement probes 4 and dummy probes 7 have been arranged in the holder 5 as shown in FIG. 3, the upper end 72a of the grip part 72 of the dummy probes 7 (the end face in the arrow Z2 direction) contacts the surface of the light blocking cloth 6 on the holder 5 side. As a result, a separation distance D10 greater than protrusion height hl is formed between the light blocking cloth 6 and the holder 5, preventing the light blocking cloth 6 from contacting the upper end 43a of the grip part 43 of the measurement probes 4.

### Configuration of Light Blocking Cloth

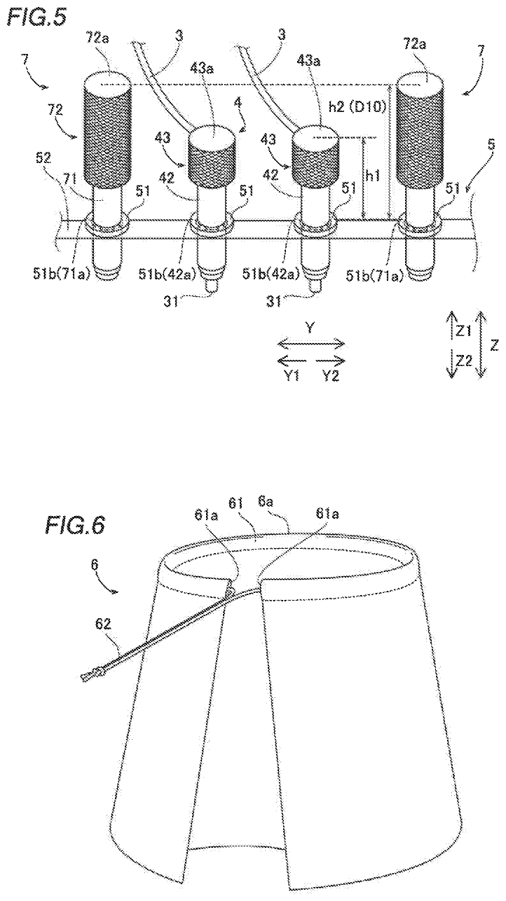

As shown in FIG. 6, in the first embodiment, the light blocking cloth 6 is made to have flexibility. Specifically, the light blocking cloth 6 is made from a material which blocks light, so as to block infrared light in particular. Consequently, the light blocking cloth 6 elastically deforms according to the state of arrangement of the dummy probes 7 and measurement probes 4 arranged in the holder 5, and can thus prevent external light (sunlight, etc.) from penetrating to the holder 5 side.

In particular, a string insertion part 61 is provided in edge 6a of light blocking cloth 6. A string member 62 is then inserted into the string insertion part 61. The two ends of the string member 62 are then pulled out through the two openings 61a of the string insertion part 61 provided at two ends of the light blocking cloth 6. The portion of the string member 62 which has been pulled out from one opening 61a and the portion of string member 62 which has been pulled out from the other opening 61a are then tied to each other.

Furthermore, the length of the edge 6a of the light blocking cloth 6 corresponds to the length of the portion of the string member 62 that has been inserted into and is inside the string insertion part 61.

Namely, the light blocking cloth 6 is designed such that the edge 6a of the light blocking cloth 6 can be shortened up by pulling the string member 62 out from the two openings 61a.

As shown in FIG. 3, the light blocking cloth 6 is put on so as to cover the entirety of the holder 5 in the state where the holder 5 has been fitted to the head of a subject P and measurement probes 4 and dummy probes 7 have been arranged in the holder 5. Furthermore, by pulling out and tying the pair of string members 62 connected to the openings 61a provided at two ends of the light blocking cloth 6, the light blocking cloth 6 is tightened up and arranged such that the edge 6a of the light blocking cloth 6 fits tightly against the forehead Pa of the subject P. In this way, the gap between the forehead Pa of the subject P and light blocking cloth 6 can be made smaller, making it possible to prevent entry of light toward the holder 5 through this gap. Furthermore, the fact that the light blocking cloth 6 has flexibility and that its edge 6a can be shortened makes it possible to suitably prevent the entry of light for subjects P with different sizes of the head and face.

Furthermore, when the holder 5 side surface of the light blocking cloth 6 contacts the dummy probes 7, a separation distance D10 corresponding to the protrusion height h2 of the dummy probes 7 is formed. Namely, the light blocking cloth 6 and measurement probes 4 do not come into contact in areas where the separation distance D10 is greater than the protrusion height hl of the measurement probes 4. It will be noted that in FIG. 3, all of the measurement probes 4 which have been arranged in the holder 5 are arranged in a state separated from (not in contact with) the light blocking cloth 6.

### Configuration of Holder

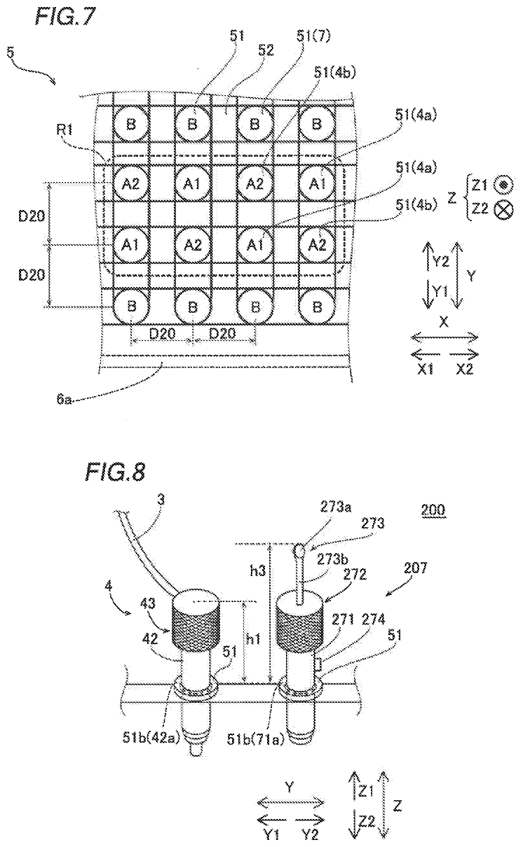

As shown in FIG. 3 and FIG. 7, the holder 5 is provided with numerous attachment parts 51 disposed in an array at a substantially equal spacing D20, and has a curved surface shape matching the shape of a head. Furthermore, the attachment parts 51 are connected to each other by means of band-shaped connecting parts 52. The connecting parts 52 are made from resin or the like, and have flexibility in the direction perpendicular to the head. It will be noted that the holder 5 shown in FIG. 3 is of a whole head type, which allows measurement probes 4 to be arranged over the entire head, and is formed in the shape of a helmet that covers the entire brain function measurement region of the head. Furthermore, the holder 5 is fastened to the head of the subject P using fastening belts (not illustrated) or the like.

The user determines the arrangement of measurement probes 4 in the attachments parts 51 (the measurement region R1) in accordance with the area (frontal, parietal, temporal, whole head, etc.) that the user wishes to measure, and attaches the measurement probes 4 to the holder 5. For example, as shown in FIG. 7, when attaching the measurement probes 4, the light sending probes 4a and light receiving probes 4b may be arranged in the respective attachment parts 51 so as to alternate in both the row and column directions. Measurement channels (measurement points) are thereby formed between adjacent light sending probes 4a and light receiving probes 4b.

In particular, as shown in FIG. 3, the attachment parts 51 have a hole 51a matching the shape of a measurement probe 4. The holder 5 is designed to allow one measurement probe 4 to be inserted into the opening 51a of each attachment part 51 and fastened therein. Specifically, as shown in FIG. 5, in the hole 51a, there is provided an engagement part 51b capable of engaging with the engagement part 42a of a measurement probe 4 or the engagement part 71a of a dummy probe 7. In particular, engagement part 51b is formed as a concave part provided in the hole 51a, and is configured so as to mate with engagement parts 42a and 71a, which are formed as convex parts.

FIG. 7 shows an example of the arrangement of measurement probes 4 and dummy probes 7 in the holder 5 according to the first embodiment. As shown in FIG. 7, looking from the arrow Z1 direction, light sending probes 4a have been arranged in a plurality of attachment parts 51 (the attachment parts 51 labeled “A1”). Light receiving probes 4b are arranged in a plurality of attachment parts 51 (the attachment parts 51 labeled “A2”). Furthermore, dummy probes 7 are arranged in a plurality of attachment parts 51 (the attachment parts 51 labeled “B”).

Namely, in the first embodiment, some of the dummy probes 7 are arranged in the holder 5 between the edge 6a of the light blocking cloth 6 and the measurement probes 4. Furthermore, in the direction along the Y axis (the direction going from the occipital region toward the frontal), dummy probes 7 are arranged on both sides of the measurement probes 4 (measurement region R1). Namely, dummy probes 7 are arranged in attachment parts 51 both on the side of edge 6a of light blocking cloth 6 (the side in the direction of arrow Y2) in the measurement region R1 formed by measurement probes 4, and on the side opposite to edge 6a of light blocking cloth 6 (the side in the direction of arrow Y1) in the measurement region R1.

The light blocking cloth 6 is thus lifted in the arrow Z1 direction by dummy probes 7 arranged both on the side of edge 6a of light blocking cloth 6 in the measurement region R1 and on the side opposite to edge 6a of light blocking cloth 6, and a separation distance D10 from the holder 5 is formed in the measurement region R1.

Furthermore, dummy probes 7 are attached to attachment parts 51 adjacent to the attachment parts 51 to which measurement probes 4 have been attached. This makes it possible to make the separation distance D10 formed in the measurement region R1 relatively large.

### Effect of the First Embodiment

The first embodiment allows the followings effects to be obtained.

In the first embodiment, as described above, the brain function measurement apparatus 100 is provided with a light blocking cloth 6 which is arranged so as to cover the holder 5 and block light from entering into the measurement probes 4 in the state where multiple measurement probes 4 have been attached to attachment parts 51; and dummy probes 7 which are arranged between the holder 5 and the light blocking cloth 6 and prevent the light blocking cloth 6 from contacting the measurement probes 4. This makes it possible to prevent light other than measurement light from entering the measurement probes 4 (light receiving probes 4b) by means of the light blocking cloth 6. Furthermore, the dummy probes 7 prevent the light blocking cloth 6 from contacting measurement probes 4 on which adjustment operations have been performed, so even if a light blocking cloth 6 is attached, the installation state of the measurement probes 4 will not change, so repeated adjustment operations on the measurement probes 4 can be avoided. As a result, because the installation state of measurement probes 4 does not change, measurement light from the measurement probes 4 can be applied accurately to the head of the subject P, and light other than measurement light can be prevented from entering the measurement probes 4 while avoiding reduction in the efficiency of measurement preparation operations.

Furthermore, in the first embodiment, as described above, the protrusion height h2 of dummy probes 7 from the holder 5 is designed to be greater than the protrusion height hl of measurement probes 4 from the holder 5 in the state where dummy probes 7 have been arranged in the holder 5. Thus, the distance between the light blocking cloth 6 and the measurement probes 4 can be increased by means of the dummy probes 7, whereof the protrusion height h2 from the holder 5 is relatively large, thus making it possible to more reliably prevent the light blocking cloth 6 from contacting the measurement probes 4.

Furthermore, the first embodiment, as described above, is designed to allow dummy probes 7 to be removably attached to attachment parts 51. Thus, there is no need to provide separate attachment parts for dummy probes 7 in order to attach dummy probes 7 to the holder 5. Furthermore, configuring the dummy probes 7 to be removably installable in attachment parts 51 makes it possible to attach the dummy probes 7 to attachment parts 51 corresponding to the optimal locations for attaching dummy probes 7 (for example, attachment parts 51 “B” in FIG. 7) in accordance with the state of arrangement of measurement probes 4.

Furthermore, in the first embodiment, as described above, the total length L11 of a dummy probe 7 is made greater than the total length L1 of a measurement probe. It is also adjustable. This allows the total length to be appropriately adjusted according to the location of arrangement of the dummy probe 7 such that the light blocking cloth 6 will not contact the measurement probes 4, thus making it possible to more effectively prevent the light blocking cloth 6 from contacting the measurement probes 4. Furthermore, if the total length L11 is made greater than that of the measurement probes 4, the protrusion height h2 of dummy probes 7 from the holder 5 can be easily made greater than the protrusion height hl of measurement probes 4 from the holder 5 by attaching dummy probes 7, which have a relatively large total length L11, to attachment parts 51.

Furthermore, in the first embodiment, as described above, the light blocking cloth 6 is formed to have flexibility and the light blocking cloth 6 is arranged such that the edge 6a of the light blocking cloth 6 fits tightly against the forehead Pa of the subject P by tying a pair of string members 62 connected to openings 61a at two ends of the light blocking cloth 6. Furthermore, dummy probes 7 are arranged on the holder 5 between the edge 6a of the light blocking cloth 6 and the measurement probes 4. Here, it is possible to bring the edge 6a of the light blocking cloth 6 into tight contact with the forehead Pa by tying two ends of the light blocking cloth 6, in order to block light other than measurement light from entering the measurement probes 4 from the direction of the forehead Pa of the subject P (arrow Y1 direction).

In this case, the closer to the edge 6a of the light blocking cloth 6, the closer the light blocking cloth 6 is arranged to the surface of the forehead Pa of the subject P, so those measurement probes 4 which are closer to the edge 6a of the light blocking cloth 6 will more readily contact the light blocking cloth 6. On this point, by configuring as described above, it becomes possible to effectively prevent the measurement probes 4 which are relatively more prone to contacting the light blocking cloth 6 from contacting the light blocking cloth 6 by means of dummy probes 7 arranged at the positions described above.

### Second Embodiment

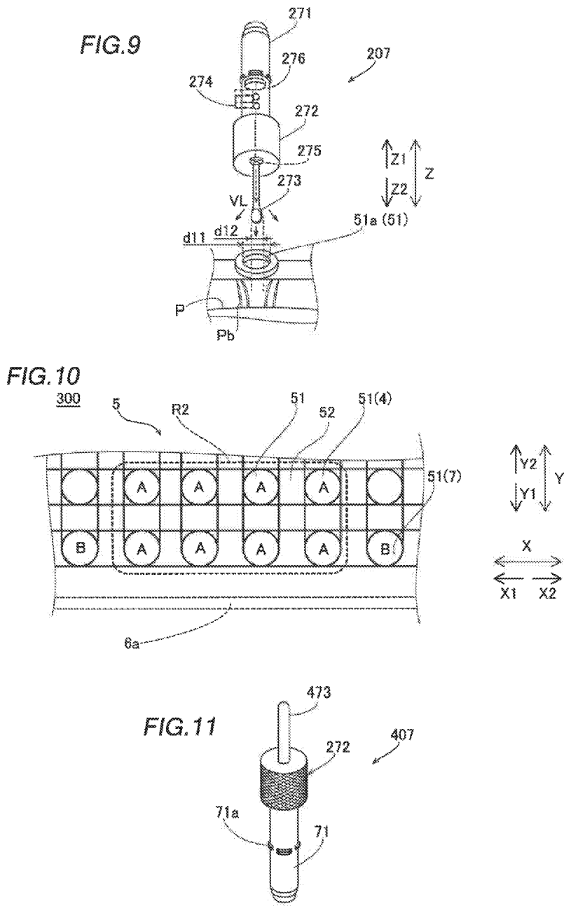

Next, the configuration of a brain function measurement apparatus 200 of a second embodiment will be described with reference to FIG. 8 and FIG. 9. In the brain function measurement apparatus 200 according to the second embodiment, the dummy probes 207 are provided with a hair parting part 273 for parting hair Pb on the head of the subject P. It will be noted that components which are the same as in the first embodiment described above will be assigned the same reference symbols in the drawings, and description thereof will be omitted. (Configuration of brain function measurement apparatus according to a second embodiment)

The brain function measurement apparatus 200 according to the second embodiment of the present invention comprises dummy probes 207 as shown in FIG. 8. A dummy probe 207 comprises a probe main body part 271, grip part 272, and hair parting part 273.

The probe main body part 271, in addition to having the configuration of probe main body part 71 of dummy probe 7 according to the first embodiment, is also provided with a switching part 274 on its side face. The grip party 272 is made substantially identical to the grip part 43 of a measurement probe 4.

Here, as shown in FIG. 9, in the second embodiment, the hair parting part 273 is designed to allow parting the hair Pb on the head of the subject P. Specifically, the hair parting part 273 is formed in the shape of a rod protruding from the grip part 272, and has a bowl-shaped tip. Namely, the hair parting part 273 has the same sort of shape as a so-called ear pick member. Furthermore, the diameter d12 of the hair parting part 273 is smaller than the diameter d11 of the hole 51a of an attachment part 51. Consequently, hair Pb can be parted by moving the hair parting part 273 inside the hole 51a of an attachment part 51.

Furthermore, in the second embodiment, dummy probe 7 comprises a light source part 275 which emits visible light VL (for example, white, green, etc.). Furthermore, the light source part 275 comprises, for example, a light emitting diode. Furthermore, dummy probe 7 comprises a battery 276 which supplies power to the light source part 275. Furthermore, switching part 274 is configured to switch between a state in which power is supplied from battery 276 to light source part 275, and a state in which power is not supplied from battery 276 to light source part 275. For example, the switching part 274 may be configured to switch between a state in which the battery 276 and light source part 275 are connected and a state in which they are not connected by being moved in the Z axis direction.

Furthermore, in the second embodiment, the hair parting part 273 of dummy probe 207 is designed to allow visible light VL to be applied from light source part 275 to the head of the subject P by guiding visible light VL from the light source part 275 in the state where the dummy probe 207 has been detached from the holder 5. Specifically, as shown in FIG. 9, the hair parting part 273 is fashioned, for example, from a member made entirely of resin or rubber and having a transparent color (having visible light transmittance). It will be noted that when the hair parting part 273 is fashioned from an elastically deformable material such as rubber, even if it should contact the head (scalp), injury to the scalp can be avoided.

Furthermore, as shown in FIG. 8, the dummy probe 207 according to the second embodiment, just as the dummy probe 7 according to the first embodiment, has a protrusion height h3 greater than the protrusion height hl of measurement probes 4 from the holder 5.

The rest of the configuration of the second embodiment is the same as in the first embodiment described above.

### Effect of the Second Embodiment

The second embodiment allows the followings effects to be obtained.

In the second embodiment, as described above, the dummy probes 207 are provided with a hair parting part 273 for parting hair Pb on the head of the subject P. A dummy probe 207 can thus be used as a hair parting member, eliminating the need to provide a member for hair parting separately from the dummy probe 207. As a result, an increase in the number of parts of the brain function measurement apparatus 200 can be avoided even though a dummy probe 207 is provided.

Furthermore, in the second embodiment, as described above, the dummy probe 207 is provided with a light source part 275 which emits light (visible light VL). Furthermore, the hair parting part 273 is designed to guide light from the light source part 275 in the state where the dummy probe 207 has been detached from the holder 5, so as to allow light from the light source part 275 to be applied to the head of the subject P. When hair Pb on the head of a subject P is to be parted, this makes it possible to apply light from the light source part 275 to the area where hair is being parted. As a result, the efficiency of the operation of parting hair Pb can be increased.

The rest of the effects of the second embodiment are the same as in the first embodiment described above.

### Modified Examples

It should be noted that the embodiments disclosed here should be seen as being in all respects illustrative and not limitative. The scope of the present invention is indicated by the scope of patent claims and not by the foregoing description of embodiments, and includes all modifications (modified example) that are of equivalent meaning to and within the scope of the patent claims.

For example, in the first embodiment and second embodiment described above, an example was presented in which the brain function measurement apparatus was provided with a portable main unit for performing brain function measurement while being carried by the subject, but the present invention is not limited to this. For example, the present invention can also be configured as a brain function measurement apparatus provided with a stationary main unit.

Furthermore, in the first embodiment and second embodiment described above, an example was presented in which the protrusion height (h2 or h3) of the dummy probes was made greater than the protrusion height h1 of the measurement probes, but the invention is not limited to this. Namely, in the present invention, the protrusion height (h2 or h3) of the dummy probes may also be made less than or equal to the protrusion height hl of the measurement probes, so long as the light blocking cloth can be prevented from contacting the measurement probes by means of the dummy probes.

Furthermore, in the first embodiment and second embodiment described above, an example was presented in which dummy probes were made installable in and removable from attachment parts in which measurement probes are installed and removed from, but the present invention is not limited to this. For example, in the present invention, dedicated attachments parts for dummy probes only may also be provided in the holder, or dummy probes (contact prevention members) may be integrally provided in the holder.

Furthermore, in the first embodiment and second embodiment described above, an example was presented in which the total length L11 of a dummy probe was made greater than the total length L1 of a measurement probe, but the invention is not limited to this. Namely, in the present invention, the total length L11 of a dummy probe may also be made less than or equal to the total length L1 of a measurement probe, so long as the light blocking cloth can be prevented from contacting the measurement probes by means of the dummy probes.

Furthermore, in the first embodiment and second embodiment described above, an example was presented in which a light blocking cloth was configured as the light blocking member, but the present invention is not limited to this. For example, a cap having light block characteristics (light blocking cap) may also be configured as the light blocking member.

Furthermore, in the first embodiment and second embodiment described above, an example was presented in which dummy probes were arranged between the edge of the light blocking cloth and the measurement probes on the holder, but the invention is not limited to this. For example, as shown for the brain function measurement apparatus 300 based on the modified example illustrated in FIG. 10, dummy probes 7 may be arranged on the holder 5 at positions (the attachment parts 51 at the positions indicated by “B” in FIG. 10) adjacent to the measurement probes 4 (the measurement probes 4 arranged at the positions indicated by “A” in FIG. 10) arranged toward the edge 6a of the light blocking cloth 6. Based on the position of arrangement of these dummy probes 7, the light blocking cloth 6 can be effectively prevented from contacting the measurement probes 4 arranged in the measurement region R2.

Furthermore, in the embodiment described above, an example was presented in which the hair parting part was formed having a shape similar to an ear pick member, but the present invention is not limited to this. For example, as shown in FIG. 11, the hair parting part 473 of the dummy probe 407 may also be formed to have rod shape with a sphere formed on its end.

Furthermore, in the embodiment described above, an example was presented in which the light source part was made from a light emitting diode, but the invention is not limited to this. For example, in the present invention, the light source part may also be made from a light bulb or from a laser diode.

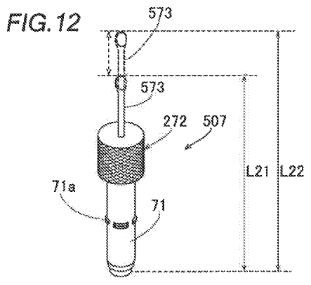

Furthermore, in the embodiment described above, an example was presented in which the dummy probe was designed to have a fixed total length, but the present invention is not limited to this. For example, it may be made variable between total length L21 and total length L22, as shown for the dummy probe 507 of the third modified example illustrated in FIG. 12. For example, the dummy probe 507 according to the third modified example comprises a hair parting part 573. The dummy probe 507 is designed to have a total length of L21 when the hair parting part 573 has been retracted and a total length of L22 when the hair parting part 573 has been extended.

What is claimed is:

1. A brain function measurement apparatus comprising: a plurality of measurement probes which either apply measurement light to the head of a subject or receive said measurement light from the head of said subject;
a holder which is fitted to the head of said subject and comprises a plurality of probe attachment parts for attaching said plurality of measurement probes;
a light blocking member which is arranged so as to cover said holder in a state where said plurality of measurement probes have been attached to said probe attachment parts, and blocks light from entering said plurality of measurement probes; and
a contact prevention member which is arranged between said holder and said light blocking member and prevents said light blocking member from contacting said measurement probes.

2. A brain function measurement apparatus according to claim 1,
wherein the height of protrusion of said contact prevention member from said holder in the state where said contact prevention member has been arranged on said holder is greater than the height of protrusion of said measurement probes from said holder.

3. A brain function measurement apparatus according to claim 1,
wherein said contact prevention member comprises dummy measurement probes which are removably attached to said probe attachment parts.

4. A brain function measurement apparatus according to claim 3,
wherein the total length of said dummy measurement probes is adjustable.

5. A brain function measurement apparatus according to claim 1,
wherein said light blocking member is formed as a flexible light blocking cloth,
said light blocking cloth is arranged such that the edge of said light blocking cloth will fit tightly against the forehead of said subject upon tying a pair of string members connected to two ends of said light blocking cloth; and
said contact prevention member is either arranged on said holder between the edge of said light blocking cloth and said measurement probes, or is arranged on said holder at a position adjacent to those of said plurality of measurement probes which are arranged toward the edge of said light blocking cloth.

6. A brain function measurement apparatus according to claim 1,
wherein said contact prevention member comprises a hair parting part for parting hair on the head of said subject.

7. A brain function measurement apparatus according to claim 6,
wherein said contact prevention member comprises a light source part which emits light; and
said hair parting part is configured to guide light from said light source part in the state where said contact prevention member is detached from said holder, thereby allowing the light from said light source part to be applied to the head of said subject.
